(19) United States
(12) Patent Application Publication
(10) Pub. No.: US 2024/0050738 A1
(43) Pub. Date: Feb. 15, 2024

(54) ACTIVE IMPLANTABLE MEDICAL DEVICE

(71) Applicant: Pacesetter, Inc., Sylmar, CA (US)

(72) Inventors: Shiloh Sison, Alameda, CA (US); Amber Durica, San Francisco, CA (US); Michael Childers, Tujunga, CA (US)

(21) Appl. No.: 18/317,605

(22) Filed: May 15, 2023

Related U.S. Application Data
(60) Provisional application No. 63/371,129, filed on Aug. 11, 2022.

(51) Int. Cl. A61N 1/08; A61B 5/00; A61B 5/055

(52) U.S. Cl. CPC A61N 1/08; A61B 5/686; A61B 5/055; A61B 2560/0242

(57) ABSTRACT

A method for automatically operating an active implantable medical device (AIMD) is provided. Under control of one or more processors, the method includes placing the AIMD in an MRI trigger mode, detecting a magnetic field of a magnetic resonance imaging (MRI) device in response to placing the AIMD in the MRI trigger mode, and communicating with a magnetic field detecting sensor to obtain characteristics of interest of the magnetic field. The method also includes determining a location of the AIMD in relation to the MRI scanner based on the characteristics of interest of the magnetic field, automatically activating an MRI mode of the AIMD, automatically deactivating the MRI mode, and maintaining the MRI trigger mode after the MRI mode is automatically deactivated.

### CROSS-REFERENCE TO RELATED APPLICATIONS

This application claims the benefit of U.S. Provisional Application No. 63/371,129 filed Aug. 11, 2022, titled “ACTIVE IMPLANTABLE MEDICAL DEVICE”, the subject matter of which is hereby incorporated by reference in its entirety.

### BACKGROUND

Embodiments of the present disclosure generally are directed towards active implantable medical devices (AIMDs) and their automatic operation in association with magnetic resonance imaging (MRI) scanners.

MRI scanners provide an effective, non-invasive imaging technique for generating sharp images of the internal anatomy of the human body, which provides an efficient means for diagnosing disorders such as neurological and cardiac abnormalities and for spotting tumors and the like. Briefly, the patient anatomy of interest is placed within the center of a large superconducting magnetic (e.g. magnetic housing) that generates a powerful static magnetic field. The static magnetic field causes protons within tissues of the body to align with an axis of the static field. A pulsed radio-frequency (RF) magnetic field is then applied causing precession of the protons around the axis of the static field. Pulsed gradient magnetic fields are then applied to cause the protons within selected locations of the body to emit RF signals, which are detected by sensors of the MRI system. Based on the RF signals emitted by the protons, the MRI system then generates a precise image of the selected locations of the body, typically image slices of organs of interest.

For patients with MR Conditional AIMDs, the AIMDs in general must be programmed or configured into an MRI mode that is compatible with MRI scanning under the prescribed conditions of use. A problem with this is that existing MRI scanning workflow requires manually programming an AIMD device into and out of MRI mode before and after a patient with an AIMD receives an MRI scan. This is a burden on users in the MRI workflow, and can also lead to human error as a result of the task not being performed property.

Accordingly, there is a need to provide an improved AIMD and method that improves upon the downfalls of manual operation of the AIMD in order to prevent human error, and reduce time-sensitive workflow steps during an MRI scanning procedure. In addition, while the AIMD operates in an MRI mode, the patient is not receiving the optimal therapy benefits of the AIMD, where delays as a result of waiting for the clinician user to manually program the AIMD out of MRI mode leaves the patient vulnerable to certain health conditions.

### SUMMARY

In accordance with embodiments herein, an active implantable medical device (AIMD) is provided that includes a magnetic field detecting sensor configured to detect characteristics of interest of a magnetic field of a magnetic resonance imaging (MRI) scanner. The AIMD also includes one or more processors, and a memory coupled to the one or more processors, wherein the memory stores program instructions. The program instructions are executable by the one or more processors to place the AIMD in an MRI trigger mode, and communicate with the magnetic field detecting sensor to obtain the characteristics of interest of the magnetic field in response to placement of the AIMD in the MRI trigger mode. The program instructions are also executable by the one or more processors to determine a location of the AIMD in relation to the MRI scanner based on the characteristics of interest of the magnetic field, and automatically activate an MRI mode of the AIMD based on the location of the AIMD in relation to the MRI scanner. The program instructions are also executable by the one or more processors to automatically deactivate the MRI mode of the AIMD based on the location of the AIMD in relation to the MRI scanner, and maintain the MRI trigger mode after the MRI mode is automatically deactivated.

Optionally, to determine the location of the AIMD in relation to the MRI scanner the one or more processors are configured to obtain a first characteristic of interest of the characteristics of interest of the magnetic field from the magnetic field detecting sensor, and associate the first characteristic of interest of the magnetic field with the location of the AIMD with respect to an MRI scanner environment. In one aspect, the first characteristic of interest is one of a first magnetic field detected along an x-axis, a second magnetic field detected along a y-axis, a third magnetic field detected along a z-axis, or a combination of at least two of the first magnetic field, the second magnetic field and the third magnetic field. In another aspect, the one or more processors are further configured to continuously monitor the characteristic of interest of the magnetic field to determine the location of the AIMD in relation to the MRI scanner while in the MRI mode, and automatically deactivate the MRI mode based on a movement of the AIMD in relation to the MRI scanner. In one example, the one or more processors are further configured to periodically check an operation of the AIMD when in the MRI trigger mode. Optionally, periodically checking operation of the AIMD includes determining a lead impedance of the AIMD or determining a pacing capture threshold communicating an alert based on a value of the lead impedance or the pacing capture threshold, and automatically deactivating the MRI trigger mode based on the value of the lead impedance or the pacing capture threshold. In another example, the one or more processors are further configured to communicate an alert based on the location of the AIMD in relation to the MRI scanner.

Optionally, the magnetic field detecting sensor is one of a Hall effect sensor or a giant magnetoresistance sensor. In one aspect, the characteristics of interest include a magnetic field of a handheld magnet for magnet reversion mode. In another aspect, the one or more processors are further configured to start a timer in response to initiating the MRI mode, and exit the MRI mode in response to a determined period of time lapsing. In one example, the one or more processors further configured to blank the MRI mode for a determined interval in response to exiting the MRI mode in response to the determined period of time lapsing. In another example, the one or more processors further configured to determine whether a handheld magnet or the MRI scanner is detected based on the characteristics of interest obtained from the magnetic field detecting sensor.

In accordance with embodiments herein, a method for automatically operating an active implantable medical device (AIMD) is provided. Under control of one or more processors, the method includes placing the AIMD in an MRI trigger mode, detecting a magnetic field of a magnetic resonance imaging (MRI) device in response to placing the AIMD in the MRI trigger mode, and communicating with a magnetic field detecting sensor to obtain characteristics of interest of the magnetic field. The method also includes determining a location of the AIMD in relation to the MRI scanner based on the characteristics of interest of the magnetic field, automatically activating an MRI mode of the AIMD, automatically deactivating the MRI mode, and maintaining the MRI trigger mode after the MRI mode is automatically deactivated.

Optionally, the method also includes obtaining a first characteristic of interest of the characteristics of interest of the magnetic field from the magnetic field detecting sensor, and associating the first characteristic of interest of the magnetic field with the location of the AIMD with respect to an MRI scanner environment. In one aspect, the method also includes automatically deactivating the MRI mode based on a movement of the AIMD in relation to the MRI scanner. In another aspect, the method additionally includes periodically checking operation the AIMD when in the MRI trigger mode. Optionally, periodically checking operation of the AIMD includes determining lead impedance of the AIMD or determining a pacing capture threshold. In one example, the magnetic field detecting sensor is one or more Hall effect sensors or a giant magnetoresistance sensor.

In accordance with embodiments herein, an active implantable medical device (AIMD) is provided that includes a first magnetic field detecting sensor configured to detect characteristics of interest of the magnetic field of a magnetic resonance imaging (MRI) device, and a second magnetic field detecting sensor configured to detect magnetic field of handheld magnet. The AIMD also includes one or more processors, and a memory coupled to the one or more processors, wherein the memory stores program instructions. The program instructions are executable by the one or more processors to place the AIMD in an MRI trigger mode, and communicate with the magnetic field detecting sensor to obtain the characteristics of interest of the magnetic field in response to placement of the AIMD in the MRI trigger mode. The program instructions are executable by the one or more processors to also determine a location of the AIMD in relation to the MRI scanner based on the characteristics of interest of the magnetic field, and automatically activate an MRI mode of the AIMD based on the location of the AIMD in relation to the MRI scanner. The program instructions are executable by the one or more processors to automatically deactivate the MRI mode of the AIMD based on the location of the AIMD in relation to the MRI scanner, and maintain the MRI trigger mode after the MRI mode is automatically deactivated. Optionally, the first magnetic field detecting sensor is a Hall effect sensor, and the second magnetic field detecting sensor is a giant magnetoresistance sensor.

### BRIEF DESCRIPTION OF THE DRAWINGS

FIG. 1A illustrates a schematic view of an active implantable medical device, in accordance with embodiments herein.

FIG. 1B illustrates a schematic view of an active implantable medical device, in accordance with embodiments herein.

FIG. 1C illustrates an example of an MRI scanner, in accordance with embodiments herein.

FIG. 2A illustrates a schematic block diagram of an automatic MRI mode module, in accordance with embodiments herein.

FIG. 2B illustrates a graph of the MRI static field detection ranges, in accordance with embodiments herein.

FIG. 3 illustrates a graph of a static field amplitude versus the distance from an isocenter of a magnetic housing of an MRI scanner, in accordance with embodiments herein.

FIG. 4A illustrates a graph of a gradient coil amplitude versus the distance from an isocenter of a magnetic housing of an MRI scanner, in accordance with embodiments herein.

FIG. 4B illustrates a graph of a gradient coil amplitude versus the distance from an isocenter of a magnetic housing of an MRI scanner, in accordance with embodiments herein.

FIG. 4C illustrates a graph of a gradient coil amplitude versus the distance from an isocenter of a magnetic housing of an MRI scanner, in accordance with embodiments herein.

FIG. 4D illustrates a graph of a gradient coil amplitude versus the distance from an isocenter of a magnetic housing of an MRI scanner, in accordance with embodiments herein.

FIG. 4E illustrates a graph of a gradient coil amplitude versus the distance from an isocenter of a magnetic housing of an MRI scanner, in accordance with embodiments herein.

FIG. 4F illustrates a graph of a gradient coil amplitude versus the distance from an isocenter of a magnetic housing of an MRI scanner, in accordance with embodiments herein.

FIG. 5 illustrates a graph of a static amplitude versus the distance from an isocenter of a magnetic housing of an MRI scanner, in accordance with embodiments herein.

FIG. 6 illustrates a block flow diagram of a process for triggering an MRI mode, in accordance with embodiments herein.

FIG. 7 illustrates a block flow diagram of the operation of an AIMD, in accordance with embodiments herein.

FIG. 8 illustrates a block flow diagram of a process for operating an AIMD in an autodetection mode, in accordance with embodiments herein.

FIG. 9 illustrates a block flow diagram of a process for operating an AIMD in an autodetection mode, in accordance with embodiments herein.

FIG. 10 illustrates block flow diagram of a process for determining between the magnet reversion, in accordance with embodiments herein.

### DETAILED DESCRIPTION

It will be readily understood that the components of the embodiments as generally described and illustrated in the Figures herein, may be arranged and designed in a wide variety of different configurations in addition to the described example embodiments. Thus, the following more detailed description of the example embodiments, as represented in the Figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

The term “characteristics of interest” shall refer to any and all types of information detected from a sensor, or determined based on information detected from a sensor. Nonlimiting examples of characteristics of interest that detecting sensor can detect, or that can be determined from signals detected by a detecting sensor include magnetic fields, changes in magnetic fields, static magnetic fields, changes in static magnetic fields, magnetic flux, radio frequency fields, change in radio frequency fields, acceleration, resistance, frequency, amplitude, or the like. The characteristics of interest can include measurements that includes units such a Gauss, Hz, Coulombs, etc., or may not have a unit of measurement.

The term “real-time” shall refer to a time period substantially contemporaneous with an event of interest, such as just prior to and/or during an MRI procedure while a patient remains stationary within an MRI scanner. The term “real-time,” when used in connection with collecting and/or processing data utilizing an AIMD, shall refer to processing operations performed substantially contemporaneous with an MRI scan of a patient. By way of example, in accordance with embodiments herein, that location of an AIMD or magnetic field detecting sensor on a table of an MRI scanner with respect to the magnetic housing of the MRI scanner are analyzed in real time (e.g., within every second, or couple of seconds while the MRI scanner is in use).

The term “MRI mode”, as used herein refers to the way, manner, state of operation, or the like of an AIMD in which one or more functionalities of the AIMD are altered, disabled, deactivated, modified, etc. as a result of the detection or presence of an MRI scanner. As an example, when the AIMD is a pacemaker, sensing can be disabled while the AIMD is in an MRI mode. The detection of the MRI scanner in one example occurs as the result of utilizing magnetic field detecting sensors, such as a three-axis Hall sensor, that detects the magnetic field, or changes in the magnetic field of the MRI scanner. In another example, the presence of an MRI scanner can be provided by a manual input from a clinician who automatically placed the AIMD into the MRI mode prior to an MRI examination.

The term “AIMD” shall mean an active implantable medical device. Embodiments may be implemented in connection with one or more active implantable medical devices (AIMDs). Non-limiting examples of AIMDs include one or more of cardiac implantable electronic devices, neurostimulator devices, implantable leadless monitoring and/or therapy devices, and/or alternative implantable medical devices. For example, the AIMD may represent a cardiac monitoring device, leaded pacemaker, cardioverter, cardiac rhythm management device, defibrillator, neurostimulator, leadless monitoring device, leadless pacemaker, and the like. The AIMD may measure electrical and/or mechanical information. For example, the AIMD may include one or more structural and/or functional aspects of the device(s) described in U.S. Pat. No. 9,333,351, entitled “NEUROSTIMULATION METHOD AND SYSTEM TO TREAT APNEA” issued May 10, 2016, and U.S. Pat. No. 9,044,610, entitled “SYSTEM AND METHODS FOR PROVIDING A DISTRIBUTED VIRTUAL STIMULATION CATHODE FOR USE WITH AN IMPLANTABLE NEUROSTIMULATION SYSTEM” issued Jun. 2, 2015, which are hereby incorporated by reference. The AIMD may monitor transthoracic impedance, such as implemented by the CorVue algorithm offered by St. Jude Medical. Additionally or alternatively, the AIMD may include one or more structural and/or functional aspects of the device(s) described in U.S. Pat. No. 9,216,285, entitled “LEADLESS IMPLANTABLE MEDICAL DEVICE HAVING REMOVABLE AND FIXED COMPONENTS” issued Dec. 22, 2015, and U.S. Pat. No. 8,831,747, entitled “LEADLESS NEUROSTIMULATION DEVICE AND METHOD INCLUDING THE SAME” issued Sep. 9, 2014, which are hereby incorporated by reference. Additionally or alternatively, the AIMD may include one or more structural and/or functional aspects of the device(s) described in U.S. Pat. No. 8,391,980, entitled “METHOD AND SYSTEM FOR IDENTIFYING A POTENTIAL LEAD FAILURE IN AN IMPLANTABLE MEDICAL DEVICE” issued Mar. 5, 2013, and U.S. Pat. No. 9,232,485, entitled “SYSTEM AND METHOD FOR SELECTIVELY COMMUNICATING WITH AN IMPLANTABLE MEDICAL DEVICE” issued Jan. 5, 2016, which are hereby incorporated by reference. Additionally or alternatively, the AIMD may be a subcutaneous AIMD that includes one or more structural and/or functional aspects of the device(s) described in U.S. application Ser. No. 15/973,195, entitled “SUBCUTANEOUS IMPLANTATION MEDICAL DEVICE WITH MULTIPLE PARASTERNAL-ANTERIOR ELECTRODES” filed May 7, 2018; U.S. application Ser. No. 15/973,219, entitled “IMPLANTABLE MEDICAL SYSTEMS AND METHODS INCLUDING PULSE GENERATORS AND LEADS” filed May 7, 2018; U.S. Application Serial Number 15/973,249, entitled “SINGLE SITE IMPLANTATION METHODS FOR MEDICAL DEVICES HAVING MULTIPLE LEADS”, filed May 7, 2018, which are hereby incorporated by reference in their entireties. Further, one or more combinations of AIMDs may be utilized from the above incorporated patents and applications in accordance with embodiments herein. Embodiments may be implemented in connection with one or more subcutaneous implantable medical devices (S-IMDs). For example, the S-IMD may include one or more structural and/or functional aspects of the device(s) described in U.S. application Ser. No. 15/973,219, entitled “IMPLANTABLE MEDICAL SYSTEMS AND METHODS INCLUDING PULSE GENERATORS AND LEADS”, filed May 7, 2018; U.S. application Ser. No. 15/973,195, entitled “SUBCUTANEOUS IMPLANTATION MEDICAL DEVICE WITH MULTIPLE PARASTERNAL-ANTERIOR ELECTRODES”, filed May 7, 2018; which are hereby incorporated by reference in their entireties. The AIMD may represent a passive device that utilizes an external power source, an entirely mechanical device, and/or an active device that includes an internal power source. The AIMD may deliver some type of therapy/treatment, provide mechanical circulatory support, and/or merely monitor one or more physiologic characteristics of interest (e.g., PAP, CA signals, impedance, heart sounds).

Additionally or alternatively, embodiments herein may be implemented in connection with an integrated healthcare patient management system or network, such as described in “METHODS, DEVICE AND SYSTEMS FOR HOLISTIC INTEGRATED HEALTHCARE PATIENT MANAGEMENT”, (Docket 13564USL1) provisional application 62/875,870, filed Jul. 18, 2019, which is incorporated by reference herein in its entirety.

Additionally or alternatively, embodiments herein may be implemented in connection with the methods and systems described in “METHOD AND SYSTEM FOR HEART CONDITION DETECTION USING AN ACCELEROMETER”, Provisional Application No. 63/021,775, which is incorporated by reference herein in its entirety.

Additionally or alternatively, embodiments herein may be implemented in connection with the methods and systems described in “METHOD AND DEVICE FOR DETECTING RESPIRATION ANOMALY FROM LOW FREQUENCY COMPONENT OF ELECTRICAL CARDIAC ACTIVITY SIGNALS”, (Docket 13964U501) (13-0396US01) U.S. application Ser. No. 16/869,733, filed on the same day as the present application, which is incorporated by reference herein in its entirety.

Additionally or alternatively, the AIMD may represent or operate in conjunction with a body generated analyte test device or “BGA test device” which represents any and all equipment, devices, disposable products utilized to collect and analyze a BGA. The AIMD may implement one or more of the methods, devices and systems described in the following publications, all of which are incorporated herein by reference in their entireties: U.S. Pat. No. 8,514,086, entitled “DISPLAYS FOR A MEDICAL DEVICE”, issued Aug. 20, 2013; U.S. Patent Publication Number 2011/0256024, entitled “MODULAR ANALYTE MONITORING DEVICE”, published Oct. 20, 2011; U.S. Patent Publication Number 2010/0198142, entitled “MULTIFUNCTION ANALYTE TEST DEVICE AND METHODS THEREFORE”, published Aug. 5, 2010; U.S. Patent Publication Number 2011/0160544, entitled “SYSTEM AND METHOD FOR ANALYSIS OF MEDICAL DATA TO ENCOURAGE HEALTHCARE MANAGEMENT”, published Jun. 30, 2011; U.S. Pat. No. 5,294,404, entitled “REAGENT PACK FOR IMMUNOASSAYS” issued Mar. 15, 1994; U.S. Pat. No. 5,063,081, entitled “METHOD OF MANUFACTURING A PLURALITY OF UNIFORM MICROFABRICATED SENSING DEVICES HAVING AN IMMOBILIZED LIGAND RECEPTOR” issued Nov. 5, 1991; U.S. Pat. No. 7,419,821, entitled “APPARATUS AND METHODS FOR ANALYTE MEASUREMENT AND IMMUNOASSAY” issued Sep. 2, 2008; U.S. Patent Publication Number 2004/0018577, entitled “MULTIPLE HYBRID IMMUNOASSAYS” published Jan. 29, 2004; U.S. Pat. No. 7,682,833, entitled “IMMUNOASSAY DEVICE WITH IMPROVED SAMPLE CLOSURE” issued Mar. 23, 2010; U.S. Pat. No. 7,723,099, entitled “IMMUNOASSAY DEVICE WITH IMMUNO-REFERENCE ELECTRODE” issued May 25, 2010; and Baj-Rossi et al. “FABRICATION AND PACKAGING OF A FULLY IMPLANTABLE BIOSENSOR ARRAY”, (2013) IEEE, pages 166-169, which are hereby incorporated by reference in their entireties.

The term “obtain” or “obtaining”, as used in connection with data, signals, information, and the like, includes at least one of i) accessing data from an internal sensor or memory of an external device or remote server where the data, signals, information, etc. are stored, ii) receiving the data, signals, information, etc. over a wireless communications link between the AIMD and a local external device, and/or iii) receiving the data, signals, information, etc. at a remote server over a network connection. The local external device may represent a clinician electronic device, an AIMD, and/or RF transceiver coupled to various other computing systems. The obtaining operation, when from the perspective of an AIMD, may include sensing new signals in real time, and/or accessing memory to read stored data, signals, information, etc. from memory within the AIMD. The obtaining operation, when from the perspective of a local external device, includes receiving the data, signals, information, etc. at a transceiver of the local external device where the data, signals, information, etc. are transmitted from an AIMD and/or a remote server. The obtaining operation may be from the perspective of a remote server, such as when receiving the data, signals, information, etc. at a network interface from a local external device and/or directly from an AIMD. The remote server may also obtain the data, signals, information, etc. from local memory and/or from other memory, such as within a cloud storage environment and/or from the memory of a workstation or clinician external programmer.

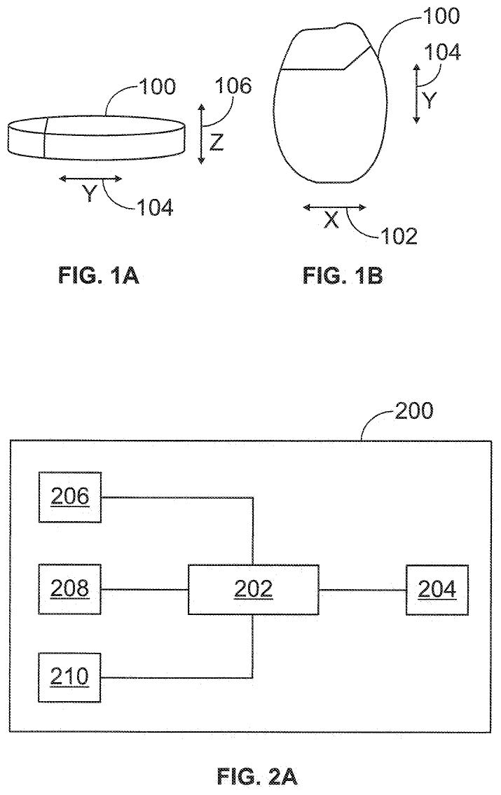

Referring now to the drawings, and in particular to FIGS. 1A and 1B, an AIMD is indicated generally at 100. Specifically, FIG. 1A is a side perspective view of AIMD 100, and FIG. 1B is a front view of AIMD 100. As shown in FIG. 1, AIMD 100 includes three axes: an x-axis 102, a y-axis 104 perpendicular to x-axis 102. and a z-axis 106 perpendicular to both x-axis 102 and y-axis 104. AIMD 100 may be, for example, a pacemaker, a cardiac resynchronization therapy defibrillator (CRT-D), an insertable cardiac monitor (ICM), a deep brain stimulation (DBS) device, a dorsal root ganglia (DRG) stimulation device, a cardiac resynchronization therapy pacer (CRT-P), or a leadless cardiac pacemaker (LCP). Alternatively, AIMD 100 may be any implantable medical device capable of functioning as described herein.

FIG. 1C illustrates an example of the MRI scanner 110. The MRI scanner 110 in one example generates an MRI RF field of 64 MHz for 1.5T, 128 MHZ for 3T and a gradient field (approximately 1 kHz). The MRI scanner 110 includes a magnetic housing 112 with an opening 114, or bore, disposed therethrough, and a movable table 116 that receives the patient. The movable table 116 moves through the opening 114 that includes the isocenter of the magnetic housing 112. The movable table 116 is of size and shape to receive a patient. The AIMD 100 when utilized in association with the MRI scanner 110 can include one or more sensors 120 for detecting the magnetic field of the MRI scanner 110.

In this example, the sensor 120 is a three-axis Hall effect sensor that includes a first magnetic field detecting sensor 124 that detects in the X-axis of the AIMD, a second magnetic field detecting sensor 126 that detects in the Y-axis of the AIMD, and a third magnetic field detecting sensor 128 the detect in the Z-axis of the AIMD. In another example, additionally, or alternatively, a fourth magnetic field detecting sensor 122 can be utilized that in one embodiment is a giant magnetoresistance (GMR) sensor. In particular, either the third magnetic field detecting sensor 128 (e.g. Z-axis Hall sensor), or the fourth magnetic field detecting sensor (e.g. GMR sensor) can be utilized for detecting lower field handheld magnets used for AIMD magnetic reversion mode.

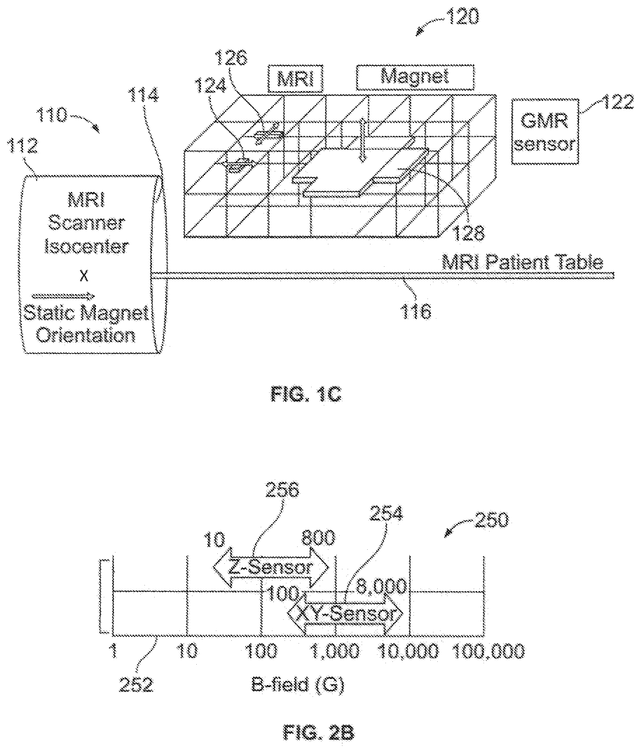

FIG. 2A is a schematic block diagram of an automatic MRI mode module 200 that may be implemented within AIMD 100 (shown in FIG. 1). Automatic MRI mode module 200 includes a processor 202 communicatively coupled to a memory device 204. Processor 202 is also communicatively coupled to detecting sensors. In one example, a first magnetic field detecting sensor 206, a second magnetic field detecting sensor 208, and a third magnetic field detecting sensor 210. In this example, the first magnetic field detecting sensor 206 is a Hall sensor that measures in the AIMD x-axis, the second magnetic field detecting sensor 208 is a Hall sensor that measures in the AIMD y-axis, and the third magnetic field detecting sensor 210 is a Hall sensor that measures in the AIMD Z-axis. To this end, a three-axis Hall sensor can be provided. In yet another example, an additional fourth detecting sensor is a GMR sensor. Although Hall sensors are described in this embodiment, those of skill in the art will appreciate that any suitable detecting sensor (e.g., a magneto resistor sensor, gradient magnetic field sensor, RF field detecting sensor, accelerometer, etc.) may be used in the systems and methods described herein. Detecting sensors 206, 208, and 210 facilitate detecting that AIMD 100 is within an MRI environment, and can automatically activate an MRI mode for AIMD 100 in response to that detection, as described herein. Processor 202 may include any suitable filtering and/or signal processing circuitry for processing signals received from the first, second, and third magnetic field detecting sensors 206, 208, and 210.

In some embodiments, executable instructions are stored in memory device 204. In the illustrated embodiment, automatic MRI mode module 200 performs one or more operations described herein by programming processor 202. For example, processor 202 may be programmed by encoding an operation as one or more executable instructions and by providing the executable instructions in memory device 204.

Processor 202 may include one or more processing units (e.g., in a multi-core configuration). Further, processor 202 may be implemented using one or more heterogeneous processor systems in which a main processor is present with secondary processors on a single chip. In another illustrative example, processor 202 may be a symmetric multi-processor system containing multiple processors of the same type. Further, processor 202 may be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein.

Processor 202 activates an MRI mode based on the MRI environment being detected by the detecting sensors 206, 208, and 210, as described herein. In the MRI mode, one or more functionalities of AIMD 100 (e.g., pacing functionality) are altered or disabled, as will be appreciated by those of skill in the art.

The memory device 204 can be one or more devices that enable information such as executable instructions and/or other data to be stored and retrieved. Memory device 204 may include one or more computer readable media, such as, without limitation, dynamic random access memory (DRAM), read-only memory (ROM), electrically erasable programmable read-only memory (EEPROM), static random access memory (SRAM), a solid state disk, and/or a hard disk. Memory device 204 may be configured to store, without limitation, application source code, application object code, source code portions of interest, object code portions of interest, configuration data, execution events and/or any other type of data.

The first, second, and third magnetic field detecting sensors 206, 208, and 210 have the capability of differentiating between magnetic fields generated by an MRI scanner and magnetic fields generated by a handheld magnet, as described herein. Further, when using a multi-dimensional Hall sensor, MRI scanner detection is possible irrespective of the physical rotation of AIMD 100 in the device pocket, which allows automatic MRI mode module 200 to accurately and reliability detect the presence of an MRI scanner.

In one example, the first and second magnetic field detecting sensors 206 and 208 (e.g. sensors 124 and 126 of FIG. 1B) can be configured to detect relatively larger magnetic fields, such as those generated by MRI scanners. In contrast, the third magnetic field detecting sensor 210 (e.g. sensor 128 of FIG. 1B) is configured to detect MRI scanner magnetic fields in addition to relatively smaller magnetic fields, such as those generated by handheld magnets. For example, in some embodiments, the third magnetic field detecting sensor 210 is capable of detecting magnetic fields greater than or equal to 10 Gauss (G), and the first and second magnetic field detecting sensors 206 and 208 are capable of detecting magnetic fields greater than or equal to 100 G. Alternatively, the magnetic field detecting sensors 206, 208, and 210 may be capable of detecting any magnetic field strength that enables AIMD 100 to function as described herein. Optionally, the first and second magnetic field detecting sensors 206 and 208 detect magnetic fields along both AIMD x-axis 102 and AIMD y-axis 104 in both polarities, while the third magnetic field detecting sensor 210 detects magnetic fields along the AIMD z-axis. Accordingly, regardless of the physical orientation of AIMD 100, first and second or first, second and third magnetic field detecting sensors 206 and 208 are able to detect the presence of an MRI scanner. Alternatively, at least one additional magnetic field detecting sensors 122 (ex. GMR sensor) can be included for detecting smaller magnetic fields (ex. Used for magnetic reversion mode) such that all four magnetic field detecting sensors 206, 208, 210 can be used for sensing MRI scanners.

As noted above, automatic MRI mode module 200 is also capable of distinguishing between fields from an MRI scanner and fields from handheld magnets. The amplitude of magnetic fields experienced by the magnetic field detecting sensors 206, 208, 210 is proportional to output voltages of the magnetic field detecting sensors 206, 208, 210 provided to processor 202. This allows AIMD 100 to avoid entering the MRI mode when only a handheld magnet is present.

Once AIMD 100 automatically detects the presence of an MRI scanner using automatic MRI mode module 200, AIMD 100 initiates programming to place AIMD 100 in the MRI mode. In one embodiment, the MRI mode lasts for a predetermined amount of time. The predetermined amount of time may be relatively short (e.g., five minutes) or relatively long (e.g., two to four hours). In some embodiments, the physician may specify the predetermined amount of time. Once the predetermined amount of time expires (i.e., after the patient has left the MRI scanner), AIMD 100 automatically returns to its default programming (e.g., a physician-recommended pacing therapy). The predetermined amount of time may be tracked using, for example, a digital timer implemented using processor 202. Accordingly, the patient does not need to visit a physician before or after the MRI procedure to have the MRI mode selectively activated and deactivated.

In another embodiment, instead of waiting for a predetermined amount of time to expire, once the MRI mode is initiated, automatic MRI mode module 200 periodically (e.g., at a rate of 8 Hz) samples the magnetic field detecting sensors 206, 208, 210 to detect the MRI scanner. Once automatic MRI mode module 200 no longer detects the MRI scanner environment for a predetermined time period, AIMD 100 returns to its default programming. This decreases the amount of time that AIMD 100 is in the MRI mode. The predetermined time period may be relatively short (e.g., five minutes) or relatively long (e.g., two to four hours).

FIG. 2B illustrates a graph 250 of the magnetic field detection ranges 252 in Gauss (G) for the AIMD X-axis (e.g. second magnetic field detecting sensor) and AIMD Y-axis (third magnetic field detecting sensor) sensors 254, and for the AIMD Z-axis sensor (first magnetic field detecting sensor) 256. In this example, the characteristics of interest of the magnetic field is the static field detection 252 are measured in G. As illustrated, the X-axis and Y-axis sensors both detect an MRI static field as small as 100G, while the Z-axis sensor detects a handheld magnet's magnetic field as small as 10G.

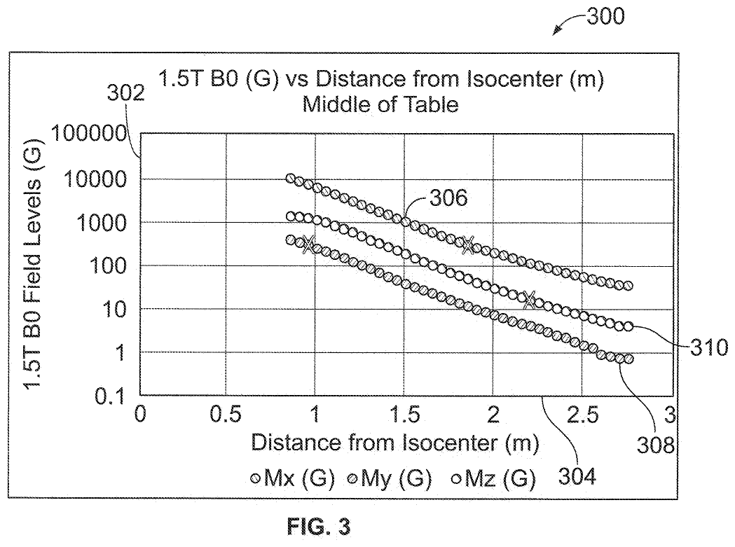

FIG. 3 illustrates a graph 300 of a 1.5T B field level 302 measured in G as a function distance 304 in meters from the isocenter of an MRI scanner. Thus, in an embodiment when a three-axis Hall sensor is utilized by the AIMD, as the AIMD moves toward or away from the isocenter, the magnetic field of the X-axis sensor, Y-axis sensor, and Z-axis sensor each varies. To this end, the magnetic field detected by the AIMD X-axis sensor (e.g. a characteristic of interest) is represented by numeral 306, the magnetic field detected by the AIMD Y-axis sensor (e.g. a characteristic of interest) is represented by numeral 308, and the magnetic field detected by the AIMD Z-axis sensor (e.g. a characteristic of interest) is represented by numeral 310. As illustrated, the magnetic field detected by each sensor correlates to a location, or distance, of the AIMD from the isocenter of the MRI scanner. This is further illustrated by table 1 (illustrating distances for a 1.5T scanner) and table 2 (illustrating distances for a 3T scanner) below using an example of a 300G detection threshold for X and Y sensors and 20G detection threshold for Z sensor:

TABLE 1

| | |
|---|---|
| | 1.5 T |
| | Distance from Isocenter When |
| Y-axis Shift of | Detection Occurs (m), Per Hall Sensor |

| Device Location | X(m) | Y(m) | X or Y(m) | Z(m) |
|---|---|---|---|---|
| Middle of Table | 1.9 | 1 | 1.9 | 2.1 |
| Left Side of | 1.8 | 1.4 | 1.8 | 2.1 |
| Table | | | | |
| Right side of | 1.8 | 1.5 | 1.8 | 1.7 |
| Table | | | | |

TABLE 2

| | |
|---|---|
| | 3 T |
| | Distance from Isocenter When |
| Y-axis Shift of | Detection Occurs (m), Per Hall Sensor |

| Device Location | X(m) | Y(m) | X or Y(m) | Z(m) |
|---|---|---|---|---|
| Middle of Table | 2.1 | 1.2 | 2.1 | 2.1 |
| Left Side of | 2.1 | 1.4 | 2.1 | 2.2 |
| Table | | | | |
| Right side of | 2.1 | 1.6 | 2.1 | 2.3 |
| Table | | | | |

Once the MRI environment is detected based on the magnetic fields (e.g. characteristics of interest) detected by the X-axis sensor, Y-axis sensor, and/or Z-axis sensor, upon the AIMD moving into the MRI environment, the MRI mode of the AIMD can be triggered. By detecting the MRI environment utilizing the magnetic field detecting sensors (e.g. Hall axes sensors) a determination can be made that the AIMD is entering the MRI scanner, and that the MRI mode should be activated.

For example, if utilizing the tables 1 and 2, the shortest distance away from the isocenter of the MRI scanner where the MRI mode triggers, or is activated, based on the X-axis sensor or Y-axis sensor is 1.8 meters from the isocenter. Gradient coil amplitudes mapped out to 0.8 meters show that the signal has attenuated by a factor of nearly ten from a maximum, where additional attenuation is expected as the patient table is moved further out by another meter. Therefore, the probability of a patient getting a foot scan, and the device not detecting the MRI static field, but instead picking up the gradient signal as cardiac activity is reduced.

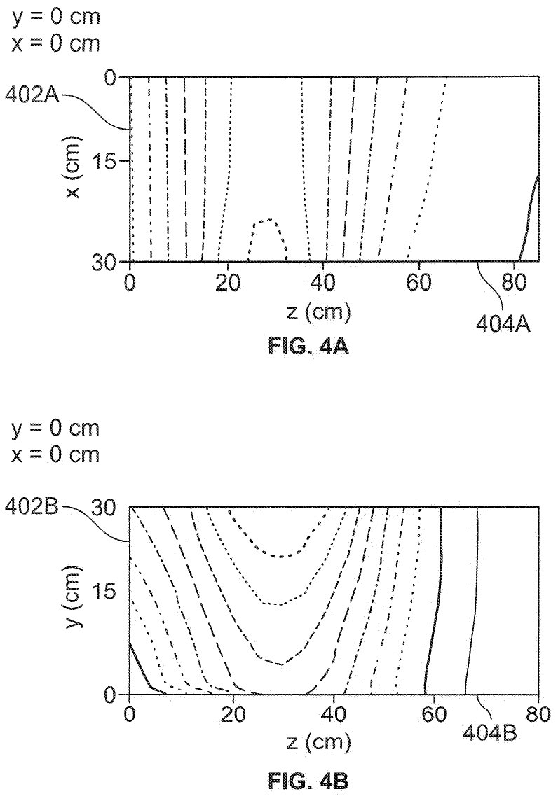

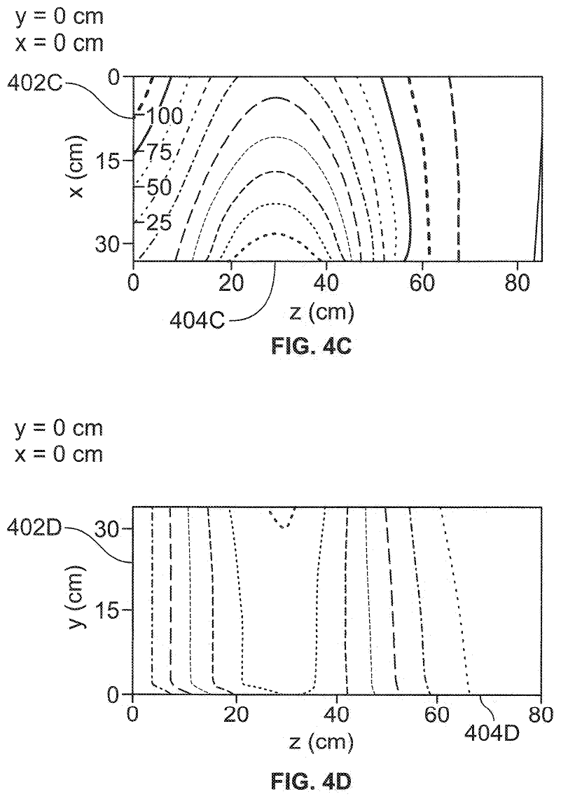

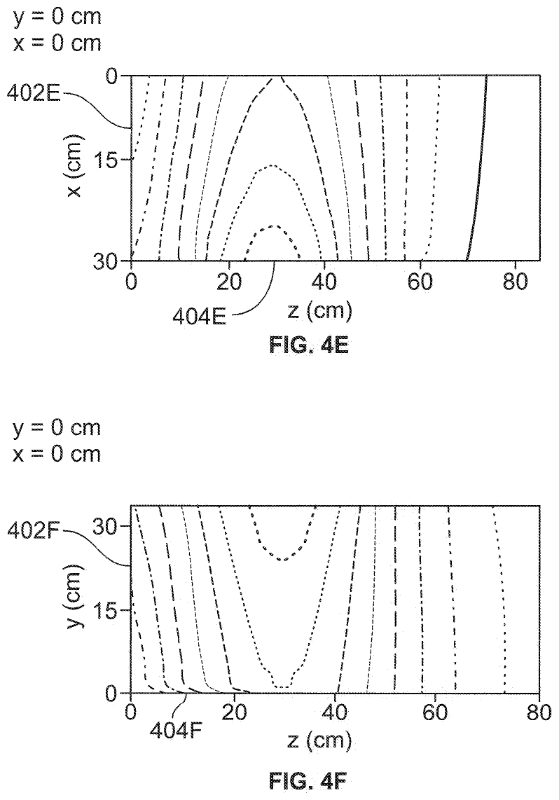

FIGS. 4A-4F show example graphs 400A-400F of gradient coil amplitude 402A-402F vs. the distance from the isocenter 404A-404F.

In addition to being able to detect the MRI environment, the magnetic field detecting sensors (e.g. Hall sensors) can also be utilized to provide measurements related to the location, or distance, of the AIMD to the isocenter. In particular, the magnetic field detecting sensors can be utilized to track an AIMD as the patient moves through and into the bore of the magnetic housing of an MRI scanner.

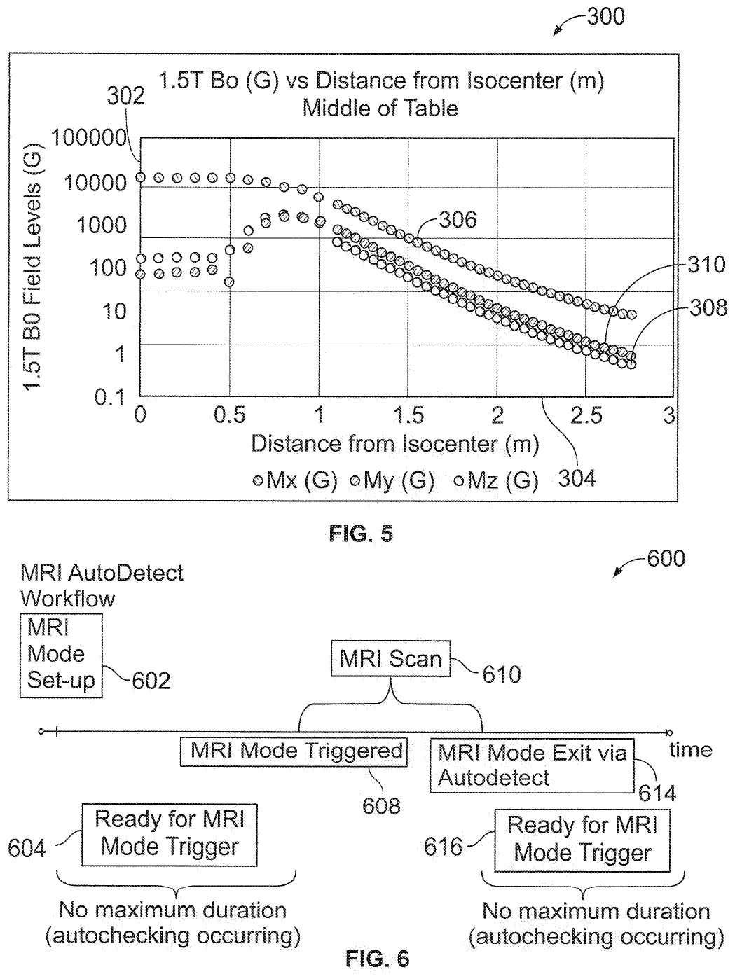

The highest component of the Bo field once the AIMD is inside the magnetic housing is the X-axis relative to the AIMD as shown in FIG. 1B. It is expected that the Y- and Z-components decrease in the isocenter of the MRI scanner. This is shown in FIG. 5. For scenario in FIG. 5, the sequence of detection thresholds as you enter the bore will be Z->X->Y->˜Y->Y. Then as you exit it will reverse if the AIMD is in the same orientation, so X, Y and Z are still on then ˜Y->Y->˜Y->˜X->˜Z. In one example, the device may be rotated in the pocket and therefore X or Y may be the sensor in-line with the highest Bo component of the MRI static field. In another example, the device may be at an angle from the plane of the table due to the shape of the pectoral region and therefore Z-axis sensor (e.g. third magnetic field detecting sensor) may additionally detect a component of the larger Mx field inside the bore. Still, a summary of example outputs is illustrated in Tables 3-5 provided below:

TABLE 3

| | | | | | | | Device | |
|---|---|---|---|---|---|---|---|---|
| | | | | | Magnet | MRI | Location | |
| State | X | Y | Z | X or Y | Reversion | Mode | in Figure 5 | Type |
| 1 | 0 | 0 | 0 | 0 | 0 | 0 | ≥2.1 m | No |
| | | | | | | | | Magnet |
| 2 | 0 | 0 | 1 | 0 | 1 | 0 | 1.9 m to | Handheld |
| | | | | | | | 2.1 m | |
| 3 | 0 | 1 | 0 | 1 | 0 | 1 | 0.4 m to | Inside |
| | | | | | | | Isocenter | Bore |
| 4 | 1 | 0 | 0 | 1 | 0 | 1 | 0.4 m to | Inside |
| | | | | | | | Isocenter | Bore |
| 5 | 0 | 1 | 1 | 1 | 0 | 1 | 1 m to 1.9 m, | Inside or |
| | | | | | | | .4 m to | Outside |
| | | | | | | | 0.75 m | Bore |
| 6 | 1 | 0 | 1 | 1 | 0 | 1 | 1 m to 1.9 m, | Inside or |
| | | | | | | | .4 m to | Outside |
| | | | | | | | 0.75 m | Bore |
| 7 | 1 | 1 | 0 | 1 | 0 | 1 | Device | Inside |
| | | | | | | | rotated | |
| | | | | | | | in pocket, in | Bore |
| | | | | | | | isocenter | |
| 8 | 1 | 1 | 1 | 1 | 0 | 1 | 0.75 m to | Inside or |
| | | | | | | | 1.1 m | Outside |
| | | | | | | | | Bore |

As illustrated, Table 3 shows a summary of sensor outputs associated with the position of the AIMD in relation to the MRI scanner isocenter. The outputs include the determined location of the AIMD based on readings of different axes of a three-axis Hall sensor as the AIMD moves toward and through the MRI environment.

Table 4 meanwhile illustrates a scenario where the patient is getting a thoracic scan and the AMID Z-axis sensor will be located around isocenter. The MRI table speeds range from 20 to 180 mm/s, therefore the durations for which the states exist as the patient table moves the AIMD Z-axis sensor into isocenter can be calculated.

TABLE 4

| | | | | | Distance | Min | Max |
|---|---|---|---|---|---|---|---|
| State | X | Y | Z | X or Y | Traveled (m) | Duration (s) | Duration (s) |
| 1 | 0 | 0 | 0 | 0 | N/A | 0 | 0 |
| 2 | 0 | 0 | 1 | 0 | .2 | 1 | 10 |
| 3 | 0 | 1 | 0 | 1 | .4 then scan | 2+ | 20+ |
| 4 | 1 | 0 | 0 | 1 | .4 then scan | 2+ | 20+ |
| 5 | 0 | 1 | 1 | 1 | .9 | 5 | 45 |
| 6 | 1 | 0 | 1 | 1 | .9 | 5 | 45 |
| 7 | 1 | 1 | 0 | 1 | .4 then scan | 2+ | 20+ |
| 8 | 1 | 1 | 1 | 1 | .4 | 2 | 20 |

Based on Table 4, for X or Y the minimum duration in each state relevant to the MRI workflow is 2 seconds.

Table 5 illustrates a summary of the state transitions and associated operation, where P is the expected probability of the transition occurring, H=High, M=Medium, L=Low. The minimum true duration in state 2 before transition is an example and may be programmable or set to a different value.

TABLE 5

| | | Minimum Duration | Transition | | |
|---|---|---|---|---|---|
| | | in State 2 Before | Magnet | | |
| State 1 | State 2 | Transition | Reversion | MRI Mode | P |
| No Magnet | Handheld | No Change from | Enter | Remain | H |
| | | existing Magnet | | Off | |
| | | Reversion | | | |
| No Magnet | Inside Bore | 2 seconds | Remain | Enter | L |
| | | | Off | | |
| No Magnet | Outside Bore | 2 seconds | Remain | Enter | M |
| | | | Off | | |
| Handheld | No Magnet | No change from | Exit | Remain | H |
| | | existing Magnet | | Off | |
| | | Reversion | | | |
| Handheld | Inside Bore | 2 seconds | Exit | Enter | L |
| Handheld | Outside Bore | 2 seconds | Exit | Enter | H |
| MRI Mode | No Magnet | Programmable | Remain | Exit | H |
| On (Inside | | with Default to | Off | | |
| Bore or | | 30 min for pacer, | | | |
| Outside Bore | | 10 min for ICD | | | |
| or Handheld | | | | | |
| Inside Bore | Handheld | 2 seconds | Remain | Remain | H |
| | | | Off | On | |
| Inside Bore | Outside Bore | 2 seconds | Remain | Remain | H |
| | | | Off | On | |
| Outside Bore | Inside Bore | 2 seconds | Remain | Remain | H |
| | | | Off | On | |
| Outside Bore | Handheld | 2 seconds | Remain | Remain | L |
| | | | Off | On | |

The low probability transitions may be used for further discriminating between MRI and non-MRI generated magnetic fields by using additional sensor outputs such as accelerometers to detect the MRI scanner patient table motion, different confirmation timing or requiring more than two states to transition before entering MRI mode. Still, Tables 1-5 each show that readings related to the characteristics of interest of the magnetic field detected by the axis sensors can be determined, and associated with the location of the sensors (and hence the patient) with respect to the isocenter of the MRI scanner. Then prior to a real time scan, these tables (e.g. associations) can be utilized to trigger an MRI mode within the AIMD. In particular, not each patient is the same size, not all scans are of the same body parts, etc. Still, magnetic field data can be obtained each time an MRI is taken and associated with the location of the sensors. In one example, such information is communicated to a remote device, cloud, etc. and stored in a storage device, memory, etc. Then, an algorithm, including an artificial intelligence algorithm can be utilized to determine and form associations, tables, reading, etc. that can be utilized in future MRI scan to trigger the MRI mode. In this manner, as more data is obtained, variables such as the height of the patient, size of the patient, body part being scanned, amplitude of sensed cardiac signals, or the like can be obtained, and utilized to determine the tables, or saved information to be utilized to determine the location of the patient and position of the device with respect to an isocenter, and whether the AIMD should be placed into an MRI mode.

FIG. 6 illustrates a work flow block diagram of a process 600 for automatically triggering an MRI mode. In one example, the MRI and AIMD assemblies and devices described in relation to FIGS. 1-5 perform the operations provided in FIG. 6. Once the MRI scanner environment is detected by the AIMD using characteristics of interest obtained by the detecting sensors, including by utilizing tables similar to tables 1-5, the AIMD can be placed into an MRI mode. In this manner, the patient along with the AIMD can be safely scanned under the MRI conditions of use. This workflow may be implemented as part of standard clinical workflow or as a user error mitigator in the event a clinician neglects to place the AIMD into the appropriate MRI mode under a more traditional workflow.

At 602, the AIMD is set in an MRI mode set-up. In an MRI mode set-up, the AIMD is set up to enable the MRI auto detection feature. At this step, the device is configured for MRI auto detection. This may include enabling of field detecting sensor(s) for monitoring characteristics of interest from the MRI environment. To this end, at 604 once the MRI mode set-up is complete, a ready for MRI trigger mode is started. Because of the autodetect capability of the AIMD, the AIMD can stay in the ready for MRI trigger mode at 604 in perpetuity or until the MRI environment is detected by the field detecting sensor(s) and MRI mode is triggered.

By providing magnetic field detecting sensors that can detect the MRI environment, the AIMD can remain in an original normal operating state up to the time the detection of the magnetic field occurs. In addition, the AIMD can also detect when the AIMD is no longer within the magnetic field of the MRI scanner, to immediately change the functioning of the AIMD back to its original normal state upon leaving the magnetic field of the MRI scanner. As a result, the AIMD is not fully functional for a minimum amount of time because of the MRI scan.

At 608 the MRI mode is triggered in response to detecting a determined characteristic of interest, by at least one field detecting sensor. In one example the AIMD may contain a three-axis Hall effect sensor where at least one axis is used to detect the MRI scanners magnetic field and trigger MRI mode. In another example the magnetic field detecting sensor is a GMR sensor. By detecting characteristics of interests, such as the changes in the magnetic field, the changes can be compared to previously recorded characteristics to determine that an AIMD is within an MRI environment. As soon as such a change in characteristics is detected, the MRI mode is automatically triggered, and at 610, prior to the MRI scan being performed. By having the MRI mode automatically trigger, clinician user does not have to manually program the MR Conditional device to MRI mode prior to scanning the patient under the MRI conditions of use.

During the MRI scanning at 610 the AIMD continues monitoring the field detecting sensor(s) confirming the device is still within the MRI environment during the scan. The AIMD is able to detect when the device is no longer in the MRI environment. Again, such determination in one example is done by comparing the changes and generated magnetic fields detected by the magnetic field detecting sensor(s) compared to previous magnetic field detecting sensor(s) determinations. In some examples, the previously captured magnetic field data includes different movements, postures, positions, or the like of the AIMD in the bore. In this manner, if a patient is laying on their side, stomach, back, etc. the AIMD is still able to determine the location of the AIMD in relation to the isocenter of the magnetic housing by determining the magnetic field detecting sensors.

Once the AIMD is determined to have exited the MRI environment, at 614, the MRI mode automatically ends or terminates.

At 616, the AIMD again operates in a ready for MRI trigger mode state. During this period the AIMD reverts back into the ready for MRI trigger state, and waits for detection of the MRI environment resulting from a new MRI scan.

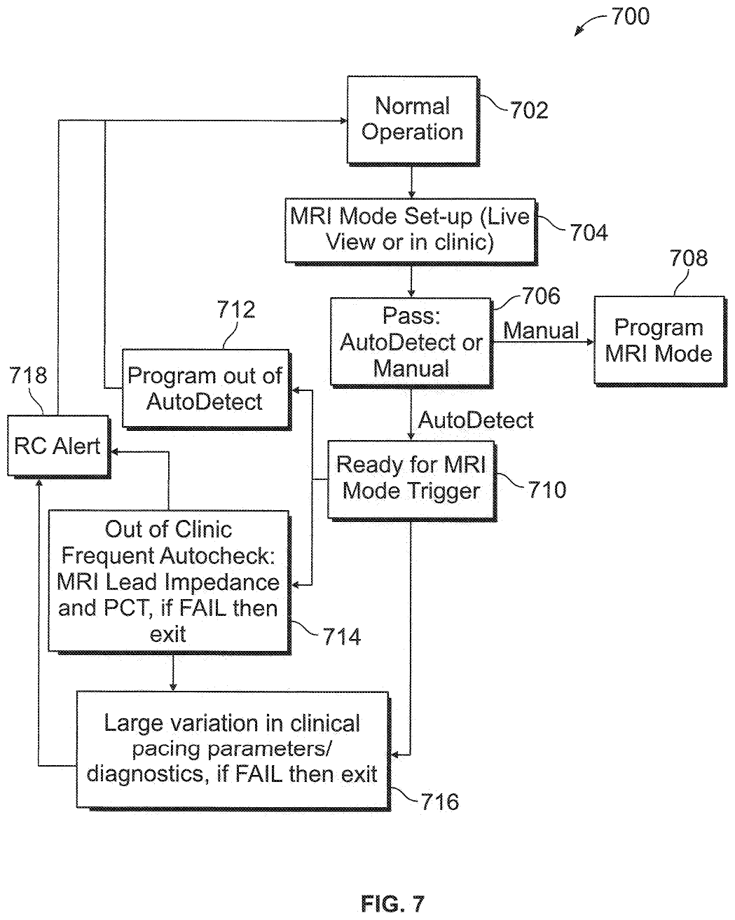

FIG. 7 illustrates a block flow diagram of the operation 700 of the AIMD by the autodetection workflow. At 702, the AIMD is operating normally. So, in an example when the AIMD is a pacemaker, the pacemaker is providing pacing accordingly. At 704, any device specific MR Conditional setting are selected and setup by the physician. This may be accomplished, for example, via remote programming or in person at a clinic or hospital. During the MRI mode set up a determination is made at 706 whether the AIMD is going to be operated in a manual setting or operational state, or in an autodetect setting or operational state. If a manual input is provided, at 708, then a clinician is responsible for manually placing the AIMD into the MRI mode, including disabling, changing, modifying, etc. functions and operations of the AIMD during an MRI scan. In this manner, the AIMD includes a mode or operating state where a clinician, technician, etc. can operate the AIMD without the use of autodetection. Providing the option of a manual mode may not be required for all devices.

Alternatively, if the AIMD is placed in an autodetect operating state or mode, then at 710, the AIMD is placed in a ready for MRI trigger mode state. Once the AIMD is placed in the autodetect mode, the AIMD can remain in the autodetect mode and does not have to go through the MRI set up mode again until, or unless the AIMD is reset to a backup state. Thus, if the AIMD had previously gone through the MRI set up mode without resetting to a backup state the AIMD, the AIMD simply remains in a ready for MRI trigger state without the need to provide an additional MRI set up mode.

Once the AIMD is in an autodetect mode or operating state and at the ready for MRI trigger mode operating state, there are several possible ways to exit the autodetect mode. First, at 712, the autodetect mode is programmed off by an input from a clinician, technician, or the like. In one example, a clinician, technician, or the like can simply remove the AIMD from of autodetect mode. In one embodiment, the mode can be changed from the autodetect mode to a manual mode

In addition, the MR Conditional device may monitor for proper device function/integrity and/or biological signals to determine if auto detection mode should be exited. For example, at 714 the AIMD conducts periodic auto checks including in one example, the AIMD lead impedance, and pacing capture threshold (PCT). In one example, at least one auto check, or verification is performed an hour. Alternatively the verification is performed once every three hours, six hours, twelve hours, twenty-four hours, thirty-six hours, forty-eight hours, etc. In all, the checks are performed with a frequency to ensure the AIMD is ready for scanning, should the need for scanning occur.

In another example, at 716 the AIMD monitors for a large variation in clinical pacing parameters or diagnostics. In either case, if a failure is detected during a verification like 714 or 716, alert may be communicated by the AIMD at 718. In one example, the alert is a failure message. This may be accomplished, for example, via remote care. In all, if the AIMD parameters or diagnostics are out of range desired for a future scan, the AIMD can programmed out of autodetect mode. In this manner, once the AIMD parameters or diagnostics return to the acceptable range, normal operation at 702 can occur again with the process restarting accordingly.

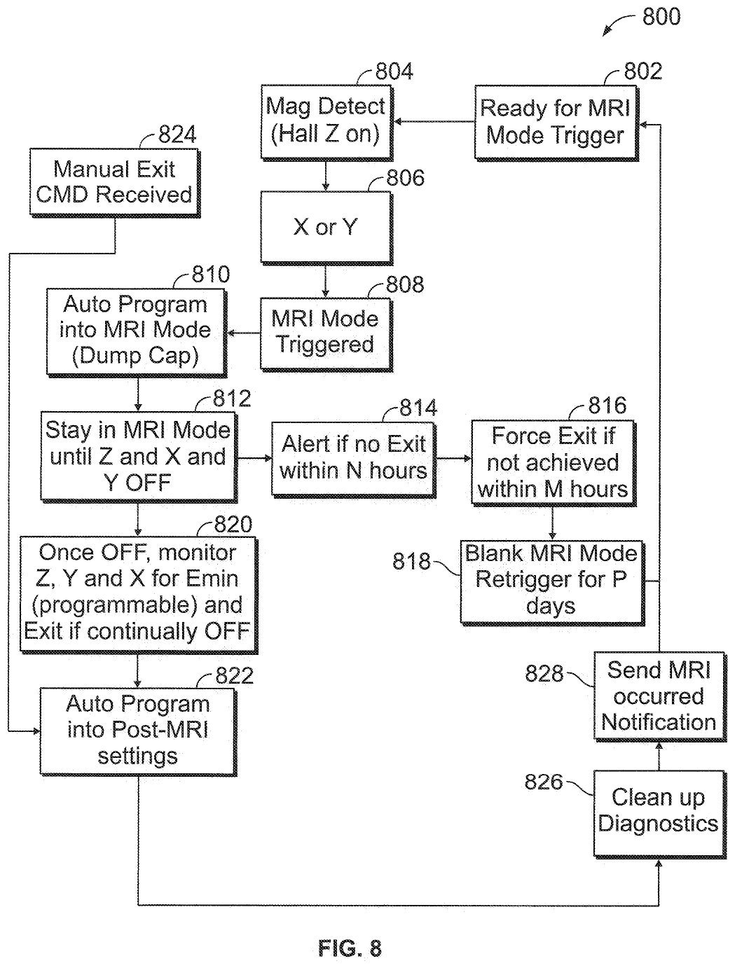

FIG. 8 illustrates block flow diagram of an autodetection process 800. The autodetection mode begins with the AIMD at 802 in the ready for MRI trigger mode. In the ready for MRI trigger mode, the AIMD continuously monitors characteristics of interest with magnetic field detecting sensors at 804 and 806. This includes for the detection of the MRI environment. In one example a first magnetic field detecting sensor 804 is a Z-axis sensor of a Hall effect sensor, and the second and third magnetic field detecting sensor 806 represents the X-axis and Y-axis sensors of a Hall effect sensor. In one example, the magnetic field detecting sensor(s) function as described in relation to at least one magnetic field detecting sensor described in relation to the embodiments of FIGS. 1-5.

Once the MRI environment is detected by the AIMD, at 808 the MRI mode is triggered at which time the AIMD prepares for and enters MRI mode at 810. In one example of device preparation, the charge stored by a capacitor is dumped prior to the MRI scanning process.

As the scanning process occurs, at 812, the AIMD continuously monitors the field detecting sensors to determine if a AIMD is still within the MRI environment and remains in MRI mode as long as the MRI environment is still detected. A timer may also be included at 814, which would result in the AIMD exiting MRI mode if a determined period of time exceeded. In one example, the determined period is an hour, two hours, four hours, six hours, etc. In particular, after a determined period, even if a AIMD remains within the MRI environment. Alternatively, in the case of an inaccurate sensor reading a AIMD could leave the MRI environment without detection by the AIMD. In either instance, if the determined period expires, at 816 the AIMD is forced to exit the MRI mode. Optionally, the AIMD communicates an alert that the timer has expired.

Optionally, upon the determined period expiring, at 818 the AIMD can begin blanking the MRI mode retrigger for a determined interval to attempt to prevent a continued error in a reading. In one example the determined interval is one day, two days, five days, a week, or the like. By blanking the retrigger, the AIMD is prevented from entering the ready for MRI trigger mode to attempt to alert a clinician, technician, etc. that the autodetect feature of the AIMD may include error, and manual operation is preferred. Still, once the determined interval is exceeded, the AIMD goes back into the ready for MRI trigger mode at 802.

If at 812, the AIMD is determined to have exited the MRI environment, then at 820 the AIMD continues monitoring for a determine period. In particular, because a chance exists that a misreading, movement by an AIMD, or the like can occur resulting in a magnetic field detecting sensor temporarily no longer detecting the AIMD, a determine period, such as ten seconds, thirty second, a minute, or the like is provided where the AIMD must continuously detect the AIMD is no longer in the magnetic housing.

Once the determined period lapses at 820, in response, at 822, the AIMD switches to post MRI settings after the AIMD has exited the MRI environment. While in one example the determined period lapses at 820 for the post MRI setting, in another example a manual input can be provided at 824 to put the AIMD in the post MRI setting. In the post MRI settings, at 826 diagnostics related to the operation of the AIMD can be cleaned. Additionally, at 828, a communication, such as an alert may be provided that the MRI scan has been completed so that the clinician, technician, etc. knows that AIMD should be operating normally. Once the notification is provided, the AIMD goes back into the ready for MRI trigger mode, and is ready to detect when the AIMD goes through another MRI environment. In this manner, the AIMD is continuously monitoring for the MRI environment without additional action by a clinician, technician, doctor, etc.

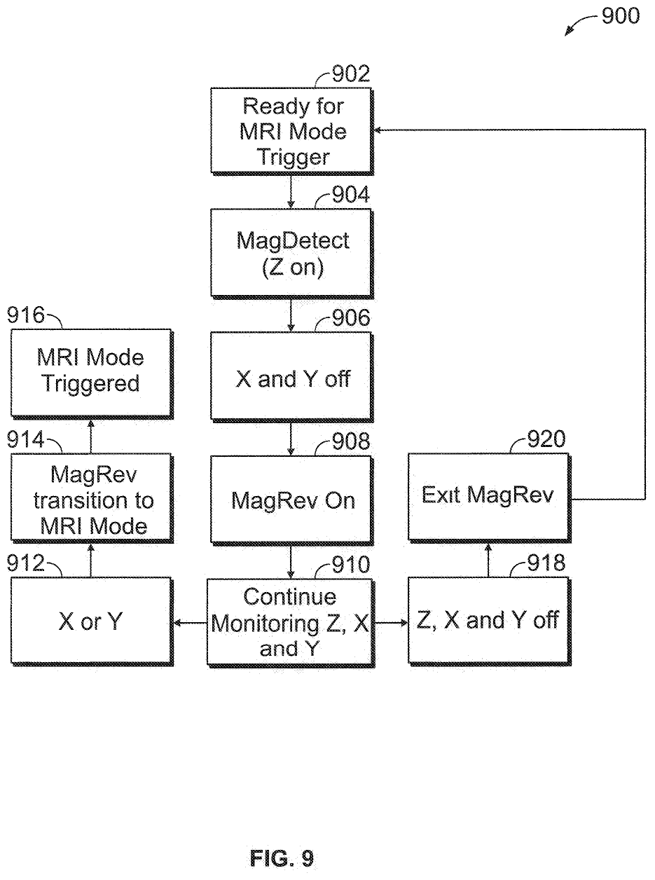

FIG. 9 illustrates a block flow diagram of a process 900 for enabling a magnet reversion mode distinct from an MRI mode. In particular, in example embodiments a magnetic field detecting sensor is used to determine if a handheld magnet has been placed over the AIMD and a magnetic reversion mode should be entered, the process 900 represents methodology for using such a determination to trigger a magnet reversion mode.

At 912, the AIMD is in a ready for MRI trigger mode. In one example the AIMD includes a first magnetic field detecting sensor that can be the Z-axis of a three-axis Hall sensor. When a determination is desired to be made to determine whether a handheld magnet is present and magnetic revision mode is needed, 904 the Z-axis detection is activated, while at 906 the X-axis, and Y-axis, used for detecting a MRI environment, are turned off. At that point, at 908, magnetic reversion mode is activated.

At 910, the AIMD continues monitoring of the X-axis, Y-axis, and Z-axis to make a determination regarding whether the AIMD should remain in magnet reversion mode because a handheld magnet is still present, exit magnet reversion mode because no magnets are present, or trigger MRI mode because the AMID has entered an MRI environment. In sum, if at 910 the Z-axis is detecting a handheld magnet and the X-axis and Y-axis are off, not detecting an MRI environment, then the AIMD will remain in magnet reversion mode. If either the X-axis or Y-axis detects the MRI environment 912, then at 914 the magnetic reversion mode transitions to an MRI mode, and the MRI mode is triggered at 916.

Alternatively, if at 910 a determination is made that no magnets are present with the X-axis, Y-axis, and Z-axis of the Hall sensor all deactivated or turned off 918, then at 920 the magnetic reversion mode of the AIMD is deactivated. In this manner, the AIMD is placed back into the ready for MRI trigger mode.

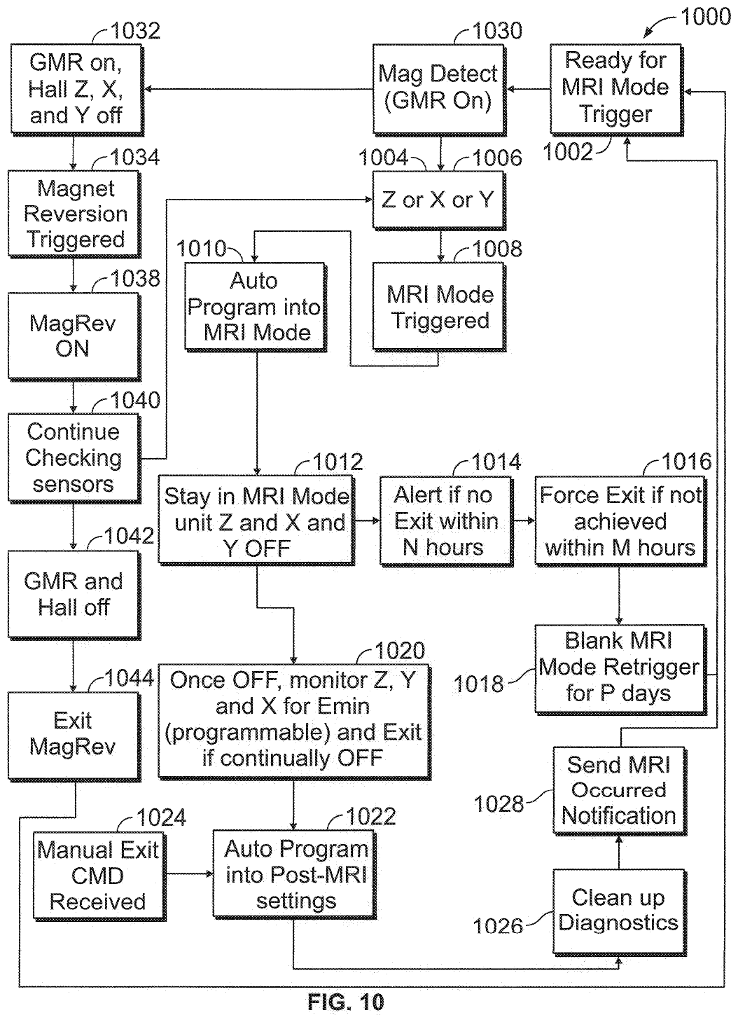

FIG. 10 illustrates an alternative block flow diagram of a process 1000 for operating an AIMD in an autodetection workflow. In the alternative embodiment an auxiliary magnetic field detecting sensor can be utilized to detect the smaller handheld magnets for magnetic reversion mode. In one such example a GMR sensor may be utilized as the auxiliary magnetic field detecting sensor.

In the embodiment of FIG. 10, the AIMD at 1002 in the ready for MRI trigger mode. In the ready for MRI trigger mode, the AIMD continuously monitors characteristics of interest with magnetic field detecting sensors at 1004 and 1006. This includes for a first magnetic field detecting sensor 1004 is a Z-axis sensor of a Hall effect sensor, and the second and third magnetic field detecting sensor 1006 represents the X-axis and Y-axis sensors of a Hall effect sensor.

At 1008 the MRI mode is triggered at which time the AIMD prepares for and enters MRI mode at 1010. In one example of device preparation, the charge stored by a capacitor is dumped prior to the MRI scanning process.

At 1012, the AIMD continuously monitors the field detecting sensors to determine if the X, Y, and Z sensors are off. A timer may also be included at 1014, which would result in the AIMD exiting MRI mode if a determined period of time exceeded. In one example, the determined period is an hour, two hours, four hours, six hours, etc. In particular, after a determined period, even if a AIMD remains within the MRI environment. Alternatively, in the case of an inaccurate sensor reading an AIMD could leave the MRI environment without detection by the AIMD. In either instance, if the determined period expires, at 1016 the AIMD is forced to exit the MRI mode. Optionally, upon the determined period expiring, at 1018 the AIMD can begin blanking the MRI mode retrigger for a determined interval to attempt to prevent a continued error in a reading. Still, once the determined interval is exceeded, the AIMD goes back into the ready for MRI trigger mode at 1002.

If at 1012, the AIMD is determined to have exited the MRI environment, then at 1020 the AIMD continues monitoring for a determine period. In particular, because a chance exists that a misreading, movement by an AIMD, or the like can occur resulting in a magnetic field detecting sensor temporarily no longer detecting the AIMD, a determine period, such as ten seconds, thirty second, a minute, or the like is provided where the AIMD must continuously detect the AIMD is no longer in the magnetic housing.

Once the determined period lapses at 1020, in response, at 1022, the AIMD switches to post MRI settings after the AIMD has exited the MRI environment. While in one example the determined period lapses at 1020 for the post MRI setting, in another example a manual input can be provided at 1024 to put the AIMD in the post MRI setting. In the post MRI settings, at 1026 diagnostics related to the operation of the AIMD can be cleaned. Additionally, at 1028, a communication may be provided that the MRI scan has been completed so that the clinician, technician, etc. knows that AIMD should be operating normally. Once the notification is provided, the AIMD goes back into the ready for MRI trigger mode, and is ready to detect when the AIMD goes through another MRI environment. In this manner, the AIMD is continuously monitoring for the MRI environment without additional action by a clinician, technician, doctor, etc.

In the example of FIG. 10 at 1030 an option can be provided to utilize the auxiliary magnetic field detecting sensor (e.g. an GMR sensor) to check for lower field handheld magnets which would trigger a magnetic reversion mode. When the auxiliary magnetic field detecting sensor, at 1030 changes to an on state detecting a handheld magnet and the X-axis sensor, Y-axis sensor, and Z-axis sensors, used to detect the MRI environment, are off, then, at 1034 the magnetic reversion is triggered and at 1038 magnetic reversion mode is activated.

At 1040 the AIMD continues monitoring of the axillary magnetic field detecting sensor (e.g. an GMR sensor) and the X-axis, Y-axis, and Z-axis to make a determination regarding whether the AIMD should remain in magnet reversion mode because a handheld magnet is still present, exit magnet mode because no magnets are present, or trigger MRI mode because the AMID has entered an MRI scanner environment. In sum, if at 1040 the axillary magnetic field detecting sensor (e.g. an GMR sensor) is detecting a handheld magnet and the X-axis, Y-axis, and Z-axis are off, not detecting an MRI scanner environment, then the AIMD will remain in magnet reversion mode. If any of the X-axis, Y-axis, or Z-axis sensors used for detecting an MRI scanner environment are activated 1004/1006, then MRI Mode will be triggered at 1008.

Alternatively at 1040, if both the auxiliary magnetic field detecting sensor, and the X-axis, Y-axis, or Z-axis—magnetic field detecting sensors are deactivated 1042. Then, at 1044, the AIMD stops operating in the magnetic reversion mode and the device returns to a ready for MRI Mode trigger state. In this manner, the auxiliary (ex. GMR) sensor can be utilized to detect a handheld magnet distinguishing it from an MRI scanner environment.

In all, a system, method, and processes are provided for using a magnetic field detecting sensor, or other appropriate sensor(s), for detecting an MRI scanner environment to automatically trigger an MRI mode of the AIMD. Functionality is provided to ensure manual operation of the AIMD is still optional, and methods and processes provided to ensure magnetic reversion mode can be triggered separate from MRI mode.

### Closing Statements

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method, or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the Figures, which illustrate example methods, devices, and program products according to various example embodiments. These program instructions may be provided to a processor of a general purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. An active implantable medical device (AIMD) comprising:
a magnetic field detecting sensor configured to detect characteristics of interest of a magnetic field of a magnetic resonance imaging (MRI) scanner;
one or more processors; and
a memory coupled to the one or more processors, wherein the memory stores program instructions, wherein the program instructions are executable by the one or more processors to:
place the AIMD in an MRI trigger mode;
communicate with the magnetic field detecting sensor to obtain the characteristics of interest of the magnetic field in response to placement of the AIMD in the MRI trigger mode;
determine a location of the AIMD in relation to the MRI scanner based on the characteristics of interest of the magnetic field;
automatically activate an MRI mode of the AIMD based on the location of the AIMD in relation to the MRI scanner;
automatically deactivate the MRI mode of the AIMD based on the location of the AIMD in relation to the MRI scanner; and
maintain the MRI trigger mode after the MRI mode is automatically deactivated.

2. The AIMD of claim 1 wherein to determine the location of the AIMD in relation to the MRI scanner the one or more processors are configured to:
obtain a first characteristic of interest of the characteristics of interest of the magnetic field from the magnetic field detecting sensor;
associate the first characteristic of interest of the magnetic field with the location of the AIMD with respect to an MRI scanner environment.

3. The AIMD of claim 2, wherein the first characteristic of interest is one of a first magnetic field detected along an x-axis, a second magnetic field detected along a y-axis, a third magnetic field detected along a z-axis, or a combination of at least two of the first magnetic field, the second magnetic field and the third magnetic field.

4. The AIMD of claim 1, wherein the one or more processors are further configured to:
continuously monitor the characteristic of interest of the magnetic field to determine the location of the AIMD in relation to the MRI scanner while in the MRI mode; and
automatically deactivate the MRI mode based on a movement of the AIMD in relation to the MRI scanner.

5. The AIMD of claim 1, wherein the one or more processors are further configured to:
periodically check an operation of the AIMD when in the MRI trigger mode.

6. The AIMD of claim 5, wherein periodically checking operation of the AIMD includes:
determining a lead impedance of the AIMD or determining a pacing capture threshold;
communicating an alert based on a value of the lead impedance or the pacing capture threshold; and
automatically deactivating the MRI trigger mode based on the value of the lead impedance or the pacing capture threshold.

7. The AIMD of claim 1, wherein the one or more processors are further configured to communicate an alert based on the location of the AIMD in relation to the MRI scanner.

8. The AIMD of claim 1, wherein the magnetic field detecting sensor is one of a Hall effect sensor or a giant magnetoresistance sensor.

9. The AIMD of claim 1, wherein the characteristics of interest include a magnetic field of a handheld magnet for magnet reversion mode.

10. The AIMD of claim 1, wherein the one or more processors are further configured to:
start a timer in response to initiating the MRI mode; and
exit the MRI mode in response to a determined period of time lapsing.

11. The AIMD of claim 10, the one or more processors further configured to:
blank the MRI mode for a determined interval in response to exiting the MRI mode in response to the determined period of time lapsing.

12. The AIMD of claim 1, the one or more processors further configured to:
determine whether a handheld magnet or the MRI scanner is detected based on the characteristics of interest obtained from the magnetic field detecting sensor.

13. A method for automatically operating an active implantable medical device (AIMD), under control of one or more processors, comprising:
placing the AIMD in an MRI trigger mode;
detecting a magnetic field of a magnetic resonance imaging (MRI) device in response to placing the AIMD in the MRI trigger mode;
communicating with a magnetic field detecting sensor to obtain characteristics of interest of the magnetic field;
determining a location of the AIMD in relation to the MRI scanner based on the characteristics of interest of the magnetic field;
automatically activating an MRI mode of the AIMD;
automatically deactivating the MRI mode; and
maintaining the MRI trigger mode after the MRI mode is automatically deactivated.

14. The method of claim 13, further comprising:
obtaining a first characteristic of interest of the characteristics of interest of the magnetic field from the magnetic field detecting sensor; and
associating the first characteristic of interest of the magnetic field with the location of the AIMD with respect to an MRI scanner environment.

15. The method of claim 13, further comprising:
automatically deactivating the MRI mode based on a movement of the AIMD in relation to the MRI scanner.

16. The method of claim 13, further comprising:
periodically checking operation the AIMD when in the MRI trigger mode.

17. The method of claim 16, wherein periodically checking operation of the AIMD includes determining lead impedance of the AIMD or determining a pacing capture threshold.

18. The method of claim 13, wherein the magnetic field detecting sensor is one or more Hall effect sensors or a giant magnetoresistance sensor.

19. An active implantable medical device (AIMD) comprising:
a first magnetic field detecting sensor configured to detect characteristics of interest of the magnetic field of a magnetic resonance imaging (MRI) device;
a second magnetic field detecting sensor configured to detect magnetic field of handheld magnet;
one or more processors; and
a memory coupled to the one or more processors, wherein the memory stores program instructions, wherein the program instructions are executable by the one or more processors to:
place the AIMD in an MRI trigger mode;
communicate with the magnetic field detecting sensor to obtain the characteristics of interest of the magnetic field in response to placement of the AIMD in the MRI trigger mode;
determine a location of the AIMD in relation to the MRI scanner based on the characteristics of interest of the magnetic field;
automatically activate an MRI mode of the AIMD based on the location of the AIMD in relation to the MRI scanner;
automatically deactivate the MRI mode of the AIMD based on the location of the AIMD in relation to the MRI scanner; and
maintain the MRI trigger mode after the MRI mode is automatically deactivated.

20. The AIMD of claim 19, wherein the first magnetic field detecting sensor is a Hall effect sensor, and the second magnetic field detecting sensor is a giant magnetoresistance sensor.
